(19) United States
(12) Patent Application Publication
(10) Pub. No.: US 2026/0337595 A1
(43) Pub. Date: Oct. 29, 2026

(54) MULTICOMPONENT DEEP EUTECTIC SOLVENTS, THEIR PREPARATION AND USE

(71) Applicant: Vio Invent Private Company, Kifisia (GR)

(72) Inventors: Thanos Andreou, Kifisia (GR); Marina Cvjetko Bubalo, Zagreb (HR); Dimitrios Kalias, Kifisia (GR)

(21) Appl. No.: 19/161,455

(22) PCT Filed: Feb. 28, 2024
(86) PCT No.: PCT/EP2024/055165

§ 371 Date: Sep. 2, 2025

(30) Foreign Application Priority Data
Mar. 1, 2023 (WO) PCT/EP2023/055248

(51) Int. Cl. C12N 9/96; C12N 9/04; C12N 9/36

(52) U.S. Cl. CPC C12N 9/96; C12N 9/0006; C12N 9/2462; C12Y 101/01001; C12Y 302/01017

(57) ABSTRACT

Methods of preparation and use of multicomponent deep eutectic solvents (DESs) include at least 2 components, at least one of them selected in the group consisting of ectoine, trimethylamine N-oxide (TMAO), sarcosine, glycerophosphocholine (GPC), dimethylsulfonopropionate (DMSP), guanidine, arginine, and taurine. Other multicomponent DESs may include at least 4 components. At least one of the components of the DESs may be selected in the group comprising naturally occurring osmolytes.

### CROSS REFERENCE TO RELATED APPLICATIONS

This present application is a § 371 national stage of International Application PCT/EP2024/055165, filed Feb. 28, 2024, which claims priority benefit of International Application PCT/EP2023/055248, filed Mar. 1, 2023, both of which are hereby incorporated herein by reference in their entireties.

### FIELD

The present invention belongs to the fields of chemistry and biotechnology, more precisely to the field of deep eutectic solvents for various uses. The invention relates to multicomponent deep eutectic solvents (DESs), methods for their preparation and use of the multicomponent DESs.

### BACKGROUND

Neoteric systems that effectively mimic the natural environment of various biological systems and of the biomolecules they consist of, i.e., any organic molecule that includes carbohydrates, protein, lipids, and/or nucleic acids, the so-called Deep Eutectic Solvents (DESs), have been in the past years intensively studied as nontoxic and highly tunable solvents in food, agrochemicals, cosmetics, and pharmaceuticals production (Vanda et al; 2019: Natural deep eutectic solvents: From their discovery to their applications. in Deep Eutectic Solvents: Synthesis, Properties, and Applications 61-81). Unlike conventional molecular solvents, a DES is a mixture of two or more, usually solid compounds combined in a suitable molar ratio to form a liquid at ambient temperature (Hansen et al., 2021: Chem. Rev. 121, 1232-1285). A particular subgroup of these systems, Natural DESs (NADESs), only consist of compounds that occur in nature (Dai et al., 2013: Anal. Chim. Acta 766, 61-68).

DESs usually have negligible volatility, are non-flammable, water-tolerant, and are easy to prepare with high purity at low price. Besides, DESs are usually considered green solvents due to their typical biodegradability and biocompatibility. Furthermore, DESs are highly tunable through varying the components or relative ratio of components and thus have a wide variety of potential applications. Currently known DESs are mostly used for extraction, but are thereafter discarded or need to be recycled. Choline chloride (ChCl) is often used due to its low price. ChCl is manufactured from ethylene oxide, which comes from ethylene, made from fossil fuels (petroleum or natural gas). For reasons of sustainability, elimination of ChCl is preferred.

Several DESs are known and reported in scientific articles and patent applications. To mention a few, patent EP3870227 relates to a deep eutectic solvent (DES) composition, comprising a combination of a glycol with a polymer solubilizer component, which polymer solubilizer component is selected from the group consisting of esters and lactones of organic acids; dicarboxylic acids; esters of dicarboxylic acids; esters, ethers and carbonates of diols and triols; and mixtures thereof, in a molar ratio of a glycol to the polymer solubilizer component in the range of between 12 to 1 and 1 to 10, preferably in a range of between 8 to 1 and 1 to 2, and more preferably in a range of between 4 to 1 and 1 to 1; the composition further comprising at least one DES constituent.

Patent application EP3971230 discloses a DES comprising:
at least one carboxylic acid which comprises at least two carboxylic acid functional groups and has a number of carbon atoms in the range of from 4 to 10;
at least one alcohol which comprises at least two alcohol functional groups, and which is selected from the group consisting of:
   alcohols having several carbon atoms in the range of from 2 to 12 carbon atoms,
   polyethylene glycol and
   polypropylene glycol; and
water in an amount of from 10 to 50 wt. % of the total weight of the DES.

Patent application EP3485874 describes a method of preparing or optimizing a system comprising a NADESs and one or more active ingredients, comprising the following steps:
a) analysing an active ingredient or target compound;
b) matching the active ingredient to at least one potential NADES starting composition;
c) synthesizing a stable, liquid NADESs-active ingredient system; and
d) optimizing the system prepared in step (c).

Patent application WO2017134190 discloses a DES consisting of (2-hydroxyethyl) trimethyl ammonium chloride and dithiothreitol in a molar ratio of from 1:2 to 1:3 and from 0% to 10% co-solvent, and methods of enzymatic production of polypeptides using the DES.

Esquembre et al. (2013; Phys. Chem. Chem. Phys., 15, 11248-11256) studied the stability of hen's egg white lysozyme in different choline chloride-based pseudo-concentrated and neat DESs has been studied by means of intrinsic fluorescence and CD spectroscopy. Thermal unfolding experiments carried out in non-diluted urea:choline chloride and glycerol:choline chloride eutectic solvents (UChCl-DES and GChCl-DES, respectively) showed the accumulation at certain temperatures of discrete, partially folded intermediates that displayed a high content of secondary structure and a disrupted tertiary structure. Reversibility of the unfolding process was incomplete in these circumstances, with the urea-based DES showing higher protein structure destabilization upon thermal treatment. On the other hand, aqueous dilution of the eutectic mixtures allowed the recovery of a reversible, two-state denaturation process. Lysozyme activity was also affected in neat and pseudo-concentrated GChCl-DES, with an increasing recovery of activity upon aqueous dilution, and full restoration after DES removal through extensive dialysis. The results of Esquembre et al. suggest that protein interactions at room temperature are reversible and depend on the DES components and on the aqueous content of the original DES dilution.

Similarly, Banerjee et al. (Adv. Healthcare Mater., 2017, 6, 1601411) observed that a DES consisting of choline:geranate solution in 1:2 ratio preserved the physical integrity of insulin while retaining its activity. Analogously, Lee et al. (J. Ind. Eng. Chem., 2018, 65, 343-348) improved human interferon stability by using sugar-based DESs. Improved stability of some other proteins, namely chemotrypsin and Trp-cage protein, at high temperatures was ensured by a choline-based DES (Yadav et al. (ACS Sustainable Chem. Eng., 2020, 8, 10151-10160; Pal et al. (J. Phys. Chem. B, 2020, 124, 7598-7610)).

Sanchez-Fernandez et al. (2022; Green Chem., 24, 4437-4442), by using lysozyme as a valuable model protein, showed the potential of choline chloride:glycerol DES (molar ratio 1:2) as synthetically accessible media for the storage and preservation of proteins in a concentrated regime without the requirement of other excipients. The behaviour of lysozyme in choline chloride:glycerol was studied over a wide range of concentrations, ranging from dilute (1.4 mg ml⁻¹, 0.100 mM) to concentrated (142 mg ml⁻¹, 9.9 mM) conditions and compared to that in aqueous buffer. It has been shown that lysozyme retained its globularity in the DES with an overall structure similar to that in aqueous phosphate buffer, which potentially enabled the protein to remain physically stable even after long periods of storage (40 days at room temperature) in the DES. Importantly, the enzymatic activity of the reconstituted and hydrated lysozyme solutions was totally retained after storage in DES.

Nardecchia et al. (2012; Biomacromolecules 13, 7, 2029-2036) report that UChCl-DES, which was obtained by heating urea and choline chloride in a 2:1 molar ratio at 80 °C and stirring until a homogeneous liquid was formed, promoted the stabilization of the collapsed state of elastin-like recombinamers—and the subsequent formation of aggregates-upon the loss of the structural water molecules involved in hydrophobic hydration. Cryo-etch scanning electron microscopy allowed the observation of these aggregates in neat DES. The suppression of the lower critical solution temperature transition, observed by differential scanning calorimetry and dynamic light scattering, confirmed the presence of the elastin-like recombinamers in their collapsed state. The transition from the collapsed to the expanded state was suppressed even after moderate aqueous dilution—for water contents ranging from 0 to ca. 45 wt %—and it was only recovered upon further addition of water—above 50 wt %. These features revealed the preferred stabilization of the collapsed state in not only neat deep eutectic solvents but also partially hydrated DES.

U.S. patent application, Pub. No. 2022/305401 discloses various combinations of two-component DESs and also suggests use of three components, albeit not specifically disclosing any combination of three components. According to this solution combinations of betaine: urea, choline chloride: urea, glycerol:betaine, glycerol:choline chloride, malic acid:choloride, lactic acid:betaine, levulinic acid:betaine, pyruvic acid:betaine, urea:betaine HCl, betaine:sorbitol, proline:levulinic acid, betaine:proline, proline:glucose, betaine:glucose, lysine:levulinic acid, sorbitol:levulinic acid, xylitol:levulinic acid, glucose:levulinic acid, glucose:levulinic acid, glycerol:sorbitol, glycerol:lactic acid, sorbitol:lactic acid, and glycerol:levulinic acid are disclosed.

In table below several disclosures of NADESs in scientific articles are compiled. The cited articles are:
1. Yancey and Somero; Biochem. J. 183, 317-323 (1979),
2. Liang, Y. et al. Bioresour. Technol. 310, 123389 (2020)
3. Zeng et al. J. Mol. Liq. 219, 74-78 (2016).
4. Benlebna, M. et al. Journal of Agricultural and Food Chemistry vol. 66 (2018).
5. Syakfanaya et al. Pharmacogn. J. 11, 267-271 (2019).
6. Jesus et al. Cryobiology 101, 95-104 (2021).
7. Panić, M. et al. Waste Manag. 120, 340-350 (2021).
8. Simeonov and Afonso, RSC Adv. 6, 5485-5490 (2016).
9. Zhong, L. et al. Bioresour. Technol. 343, 126022 (2022).
10. Pedro, S. N. et al. Pharmaceutics 14, 827 (2022).
11. Karadendrou, et al. Catalysts 12, (2022).
12. Sut, S. et al. Molecules 22, 1-11 (2017).
13. Faggian, M. et al. Molecules 21, 1-11 (2016).
14. Ilgen and König Green Chem. 11, 848-85 (2009).
15. Zdanowicz Int. J. Biol. Macromol. 176, 387-393 (2021).
16. Wang, T. et al. J. Pharm. Biomed. Anal. 145, 339-345 (2017)

TABLE 1
Reported deep eutectic solvents comprising
naturally occurring osmolytes

| | | | | Water | |
|---|---|---|---|---|---|
| Components | | | Components | content | |
| 1 | 2 | 3 | molar ratio | (wt. %) | Reference |
| betaine | urea | — | 1:1:2_{(H2O)} | 17 | 3 |
| betaine | glycerol | — | 1:2:2_{(H2O)} | 10 | 4 |
| betaine | sorbitol | — | 1:1.2:6_{(H2O)} | 25 | 5 |
| betaine | trehalose | — | 4:1 | unk. | 1 |
| betaine | xylose | — | 2:1:3_{(H2O)} | 12 | 6 |
| betaine | glucose | — | 5:2 | — | 1 |
| betaine | mannose | — | 5:2 | unk. | 1 |
| betaine | lysine | — | 1:1:27-180_{(H2O)} | 65-93 | 2 |
| betaine | histidine | — | 1:1:28-190_{(H2O)} | 65-93 | 2 |
| betaine | arginine | — | 1:1:30-200_{(H2O)} | 65-93 | 2 |
| glucose | fructose | — | 1:1:8_{(H2O)} | 30 | 7 |
| glucose | glycerol | — | 1:4 | — | 6 |
| glucose | trehalose | — | 1:2:13_{(H2O)} | 21 | 6 |
| fructose | trehalose | — | 1:2:13_{(H2O)} | 20 | 6 |
| glycerol | trehalose | — | 30:1 | — | 6 |
| glycerol | urea | — | 5:2 | unk. | 8 |
| glycerol | guanidine | — | 5:2 | — | 9 |
| | HCl | | | | |
| glycerol | arginine | — | 4:1 | — | 10 |
| proline | glucose | — | 5:3 | unk. | 1 |
| proline | glycerol | — | 1:2 | — | 11 |
| proline | sorbitol | — | 1:1 | unk. | 1 |
| proline | sucrose | — | 2:1, 3:1 | unk. | 1 |
| proline | urea | — | 2:1 | unk. | 12 |
| proline | glutamic | — | 2:1 | unk. | 13 |
| | acid | | | | |
| serine | glucose | — | 5:4 | unk. | 1 |
| carnitine | urea | — | 2:3 | — | 14 |
| betaine | sorbitol | urea | 1:1.2:1:7_{(H2O)} | 25 | 5 |
| betaine | trehalose | raffinose | 9:1:1:35_{(H2O)} | 25 | 1 |
| betaine | sucrose | proline | 5:2:2:21_{(H2O)} | 20 | 6 |
| betaine | glycerol | sucrose | 2:3:1:5_{(H2O)} | 10 | 6 |
| betaine | glycerol | trehalose | 2:3:1:5_{(H2O)} | 13 | 6 |
| betaine | sucrose | proline | 5:2:2:21_{(H2O)} | 20 | 6 |
| sucrose | glucose | fructose | 1:1:1:11_{(H2O)} | 22 | 6 |
| fructose | glucose | trehalose | 1:1:1:11_{(H2O)} | 21 | 6 |
| fructose | glucose | urea | 1:1:2 | unk. | 15 |
| fructose | glycerol | urea | 2:1:1 | — | 15 |
| glucose | glycerol | urea | 2:1:1 | | 15 |
| glycerol | sucrose | sorbitol | 2:1:2:10_{(H2O)} | 16 | 6 |
| glycerol | trehalose | sorbitol | 2:1:2:10_{(H2O)} | 16 | 6 |
| glycerol | glucose | sorbitol | 1:1:1:3_{(H2O)} | 12 | 6 |
| sorbitol | glycerol | urea | 1:1:2 | — | 16 |
| trehalose | glucose | sorbitol | 1:2:1:13_{(H2O)} | 17 | 6 |
| proline | fructose | glycerol | 1:1:1:5_{(H2O)} | 20 | 16 |
| proline | glucose | glycerol | 5:3:3:20_{(H2O)} | 21 | 6 |

Unk. = unknown.

### SUMMARY

Embodiments disclosed herein provide alternative DESs that will extend the use of these systems in various fields and will eliminate choline chloride. The disclosed embodiments satisfy emergent needs for green solvents with diverse properties in the pharmaceutical industry, biochemistry, material science and biotechnology. Preferably, the embodiments disclosed can provide novel DESs, without ChCl, that could also be used in final products and would exhibit novel properties and functions.

The multicomponent DESs according to a first aspect comprises at least 2 components, wherein at least one of the components is selected in the group consisting of ectoine, trimethylamine N-oxide (TMAO), sarcosine, glycerophosphocholine (GPC), dimethylsulfonopropionate (DMSP), guanidine, arginine, and taurine. Preferably, the multicomponent DESs has 3, more preferably 4 components. Optionally, the DESs also comprises water. It has been observed that these multicomponent DESs improve biomolecule stability, as well as stability and viability of cells stored at lower temperatures, while maintaining the biomolecule function or even improving it. Consequently, a possible embodiment is also a medium for stabilization of biomolecules or cryoprotection of cells/tissues comprising the DESs comprising at least two components, wherein at least one of the components is selected in the group consisting of ectoine, TMAO, sarcosine, GPC, DMSP, guanidine, arginine and taurine. A method for stabilization of biomolecules or for of biological systems, such as organisms, organs, organelles, cells or tissues comprises at least a step in which the biomolecule or biological systems, cells or tissues or artificial engineered constructs are exposed to the above-mentioned medium. The medium 5 may be supplemented with any other components and storage of the mixture of medium and biomolecules, biological systems, cells or tissues is performed in any suitable manner. The term preservation relates to any kind of processes for storing biological systems or biomolecules that aims to maintain usual biological, chemical and/or physical properties of the biological system or biomolecules, respectively. The term preservation may relate to storage at any given temperature, including cryopreservation at low temperatures, typically in the range from −80 °C to −196 °C, or preservation at temperatures above −80 °C.

A second aspect showing the same technical effect as the DESs described above is a multicomponent DESs comprising at least 4 components. In a preferred embodiment of the multicomponent DESs comprising at least 4 components, at least one of the components of the DESs are selected in the group comprising naturally occurring osmolytes. Therefore, another possible embodiment is also a medium for stabilization of biomolecules or preservation, for example cryoprotection, of biological systems, cells or tissues comprising the DESs comprising at least four components. A method for stabilization of biomolecules or for preservation/cryoprotection of biological systems, cells or tissues comprises at least a step in which the biomolecule or cells/tissue are exposed to the above-mentioned medium. The medium may be supplemented with any other components and storage of the mixture of medium and biomolecules, biological systems, cells or tissues is performed in any suitable manner.

Optionally, the naturally occurring osmolytes are selected in the group comprising:
(i) polyols and sugar polyols, for example glycerol, sorbitol, xylitol;
(ii) sugars and their derivatives, for example glucose, sucrose, trehalose;
(iii) amino acids and their derivatives, for example glycine, proline, ectoine, taurine;
(iv) methylamines, for example trimethylamine N-oxide (TMAO), sarcosine, betaine, glycerophosphocholine (GPC);
(v) methylsulfonium compounds, for example dimethylsulfoniopropionate (DMSP);
(vi) Y-conjugated compounds, for example urea, guanidine and arginine.

Preferably, the naturally occurring osmolytes are selected in the group consisting of ectoine, TMAO, sarcosine, GPC, DMSP, guanidine, arginine, and taurine. The DESs may optionally comprise water.

The other remaining components of the multicomponent DESs can be selected among common DES/IL compounds, such as methylamines, methylsulfonium compounds, organic acids, sugars, polyols, amino acids and Y-conjugated compounds. As mentioned before, in the method or used defined herein, the DES may be formulated with or without water, such as solution comprising a DES essentially free of water.

Methylamines may be selected from trimethylamine N-oxide (TMAO), betaine, glycerophosphocholine (GPC), carnitine, homarine, and derivatives thereof, for example, their halide forms, such as betaine halides (betaine HCl).

Methyl sulfonium compounds may be selected from dimethylsulfonopropionate (DMSP) and other compounds containing a methylsulfonium moiety.

Organic acids may be selected from levulinic acid, lactic acid, malic acid, maleic acid, pyruvic acid, fumaric acid, succinic acid, citric acid, citraconic acid, glutaric acid, glycolic acid, acetic acid, aconitic acid, tartaric acid, ascorbic acid, malonic acid, oxalic acid, glucuronic add, neuraminic acid, sialic acid, shikimic acid, phytic acid, galacturonic acid, iduronic acid, hyaluronic acid, hydroxycitric acid, lactone derivatives and derivatives thereof.

Sugars may be selected from trehalose, glucose, sucrose, lactose, ribose, galactose, fructose, etc. and derivatives thereof.

Polyols may be selected from glycerol, erythritol, mannitol, sorbitol, xylitol, ethylene glycol, propylene glycol, ribitol, aldonitol, propanediol, inositol, pentylene glycol, and derivatives thereof (such as o-methyl-inositol).

Amino acids may be selected from glycine, proline, taurine, lysine, etc. and derivatives thereof (e.g. ectoine, sarcosine, theanine, dimethyglycine, etc.).

Y-conjugated compounds may be selected from urea, guanidine, arginine and compounds that incorporate the guanidino moiety (such as creatine, glycocyamine, agmatine, 4-guanidinobutanoic acid etc.) and derivatives thereof, such as guanidinium halides (for example guanidine HCl).

According to another aspect, the molar ratio of each DES component with respect to the total moles of the DES may range from 0.001 to 0.9. Molar ratio is defined as unit of the amount of a component (expressed in moles), nᵢ, divided by the total amount of all components in a mixture (also expressed in moles), nₜₒₜ. In a preferred embodiment, the molar ratio of each DES component with respect to the total moles of the DES may be between 0.003 and 0.7. In a further preferred embodiment, the molar ratio of each DES component with respect to the total moles of the DES may be between 0.005 and 0.6. The preferred ratios may also depend on the combination of components in the DES as well as on the targeted use of the DES.

As mentioned before, the DES may be formulated with or without water (such as solution comprising a DES wherein the solution is essentially free of water). The water used in the DESs may be from 0% to 90%, as from about 0.00001, 0.0001, 0.001, 0.01, 0.1, 0.5, 1, 5, 10, 15, 20, 30 or about 40% to about 90, 80, 70, 60 or about 50%. In a preferred embodiment, the water used in from about 5% to about 30%, such as about 10%, 20% 25% or about 30%. The DESs as prepared may also be further diluted.

In the most preferred embodiments the multicomponent DESs comprise the components in combinations as given in the Tables 2 and 3 below.

TABLE 2
Preferred embodiments of the multi-component deep eutectic
solvents comprising naturally occurring osmolytes

Components

| 1 | 2 | 3 | Water |
|---|---|---|---|
| betaine | arginine | — | yes |
| betaine | guanidine HCl | — | yes |
| TMAO | urea | — | yes |
| TMAO | glycerol | — | yes |
| TMAO | sorbitol | — | yes |
| TMAO | glucose | — | yes |
| TMAO | trehalose | — | yes |
| TMAO | xylitol | — | yes |
| TMAO | guanidine HCl | — | yes |
| TMAO | arginine | — | yes |
| sarcosine | urea | — | yes |
| sarcosine | guanidine HCl | — | yes |
| sarcosine | arginine | — | yes |
| sarcosine | glycerol | — | yes |
| GPC | urea | — | yes |
| GPC | guanidine HCl | — | yes |
| GPC | arginine | — | yes |
| DMSP HCl | urea | — | yes |
| DMSP HCl | guanidine HCl | — | yes |
| DMSP HCl | arginine | — | yes |
| DMSP HCl | glycerol | — | yes |
| glycerol | sorbitol | — | no |
| proline | guanidine HCl | — | yes |
| proline | arginine | — | yes |
| ectoine | urea | — | yes |
| ectoine | guanidine HCl | — | yes |
| ectoine | arginine | — | yes |
| ectoine | glycerol | — | yes |
| sorbitol | urea | — | yes |
| sorbitol | guanidine HCl | — | yes |
| sucrose | urea | — | yes |
| sucrose | guanidine HCl | — | yes |
| sucrose | arginine | — | yes |
| betaine | sorbitol | guanidine HCl | yes |
| betaine | taurine | glycerol | no |
| betaine | ectoine | glycerol | no |
| betaine | ectoine | sorbitol | yes |
| TMAO | urea | glycerol | yes |
| TMAO | guanidine HCl | glycerol | yes |
| TMAO | urea | sorbitol | yes |
| TMAO | guanidine HCl | sorbitol | yes |
| TMAO | betaine | urea | yes |
| TMAO | betaine | guanidine HCl | yes |
| fructose | glucose | guanidine HCl | yes |
| fructose | glycerol | guanidine HCl | yes |
| glucose | glycerol | guanidine HCl | yes |
| glucose | trehalose | urea | yes |
| glucose | trehalose | guanidine HCl | yes |
| sorbitol | glycerol | guanidine HCl | no |
| trehalose | glucose | glycerol | no |
| trehalose | glucose | sorbitol | yes |
| trehalose | proline | glycerol | yes |
| trehalose | ectoine | glycerol | yes |
| proline | glycerol | sorbitol | yes |

TABLE 3
Preferred embodiments of the multi-component deep
eutectic solvents comprising 4 or more components

| | | | | | | | Water |
|---|---|---|---|---|---|---|---|
| Components | | | | | | | content |
| 1 | 2 | 3 | 4 | 5 | 6 | Components molar ratio | (wt. %) |
| Betaine | GPC | Sorbitol | Taurine | Urea | — | 1:2.8:3.1:0.1:7.1:1_{(H2O)} | 7 |
| Betaine | GPC | Sorbitol | Inositol | Urea | — | 1:0.7:0.7:0.9:7.1:3.5_{H2O)} | 38 |
| Betaine | GPC | Sorbitol | Inositol | Urea | — | 1:0.9:1.2:0.6:8.1:3_{(H2O)} | 30 |
| Betaine | GPC | Sorbitol | Inositol | Urea | — | 1:0.9:1.5:0.4:8.3:2.5_{(H2O)} | 21 |
| Betaine | Glycerol | Sorbitol | Ectoine | — | — | 1:4:2:1:14_{(H2O)} | 20 |
| TMAO | Betaine | Taurine | Urea | — | — | 1:0.1:0.1:1.5:5_{(H2O)} | 34 |
| TMAO | Betaine | Taurine | Urea | — | — | 1:6:0.5:15:9_{(H2O)} | 9 |
| TMAO | Betaine | Sorbitol | α-alanine | Taurine | Urea | 1:0.1:0.05:0.1:0.05:2:2_{(H2O} | 14 |
| TMAO | Betaine | Inositol | α-alanine | Urea | — | 1:0.1:0.05:0.2:1:5_{(H2O)} | 37 |
| TMAO | Inositol | Urea | α-alanine | — | — | 1:0.05:1:0.2:5_{(H2O)} | 37 |
| Trehalose | Proline | Glycerol | Sorbitol | — | — | 1:1:4:1:14_{(H2O)} | 20 |

DESs according to some embodiments are prepared with a method comprising the following steps:
a) mixing components in required molar ratios, and
b) stirring and heating the mixtures up to 60 °C until a colourless and homogeneous liquid is formed.

Use of the multi-component DESs can be multi-faceted, wherein at least the following uses are preferred:
stabilization of biomolecules,
preservation of biological systems, including cryoprotection,
pharmaceutical preparations, such as pharmaceutical excipients and similar components,
cosmetics,
food, and
preparation of extracts, wherein the DESs is optionally a component of the final extract.

Use of DES systems disclosed herein is possible for the extraction, stabilization, analysis, formulation, preservation and/or any form of culture, monitoring, handling, treatment and use of organisms, organs, tissues, organelles, biological systems, cells, biomolecules and bioactive compounds, including but not limited to plants, animals, algae, bacteria, fungi and other microorganisms, whole cells, membranes and other cell components, proteins, enzymes, antibodies, peptides and other molecules consisting of aminoacids or aminoacid derivatives, polysaccharides and biopolymers of any composition, lipids and supramolecular structures containing lipids, nucleotides, nucleosides and their precursors and derivatives, polynucleotides of any length, sense and molecular weight and composition, including but not limited to all types of DNA and RNA, all types of gels, emulsions, dispersions, liquid and/or solid compositions comprising the DESs according to disclosed embodiments and/or biomolecules and/or other payloads, including all types of bioactive compounds.

The preparations comprising the DESs as disclosed herein may be used as pharmaceutical excipients, cosmetic compositions, nutritional compositions, feed compositions, biomass extracts, nutritional products, etc.

An aspect is thus a stabilizing composition comprising any of the DESs described above for use in stabilization of biomolecules, particularly proteins, most preferably enzymes. Another aspect is a composition or medium for preservation, for example cryoprotection of biological systems, cells, tissues and molecules comprising any of the DESs described above. The medium may be supplemented with any other components and storage of the mixture of medium and biomolecules, biological systems, cells or tissues is performed in any suitable manner. An additional aspect is also the method for stabilization of biomolecules or for preservation/cryoprotection of biological systems, cells or tissues, the method comprising at least a step in which the biomolecule, biological systems, cells or tissue are exposed to the above-mentioned medium.

### DETAILED DESCRIPTION

In the most preferred embodiments the multicomponent DESs comprise the components in combinations and ratios as given in the Table 4 below.

TABLE 4
Exemplary embodiments of the multi-component deep eutectic
solvents comprising naturally occurring osmolytes

| | | | | Water |
|---|---|---|---|---|
| Components | | | Components | content |
| 1 | 2 | 3 | molar ratio | (wt. %) |
| Betaine | arginine | — | 5:1:28_{(H2O)} | 40 |
| Betaine | guanidine HCl | — | 1:2:3_{(H2O)} | 17 |
| TMAO | urea | — | 1:1:3.5_{(H2O)} | 33 |
| TMAO | glycerol | — | 1:1:2_{(H2O)} | 17 |
| TMAO | sorbitol | — | 1:1:2_{(H2O)} | 12 |
| TMAO | glucose | — | 5:2:12_{(H2O)} | 23 |
| TMAO | trehalose | — | 4:1:8_{(H2O)} | 18 |
| TMAO | xylitol | — | 1:1:4_{(H2O)} | 24 |
| TMAO | guanidine HCl | — | 1:1:2_{(H2O)} | 18 |
| TMAO | arginine | — | 15:4:56_{(H2O)} | 37 |
| sarcosine | urea | — | 2:5:9_{(H2O)} | 25 |
| sarcosine | guanidine HCl | — | 2:5:11_{(H2O)} | 23 |
| sarcosine | arginine | — | 3:2:19_{(H2O)} | 36 |
| sarcosine | glycerol | — | 1:2:4_{(H2O)} | 20 |
| GPC | urea | — | 1:2:1.5_{(H2O)} | 7 |
| GPC | guanidine HCl | — | 1:2:1_{(H2O)} | 4 |
| GPC | arginine | — | 11:4:80_{(H2O)} | 30 |
| DMSP HCl | urea | — | 1:2:2_{(H2O)} | 11 |
| DMSP HCl | guanidine HCl | — | 1:1:3_{(H2O)} | 17 |
| DMSP HCl | arginine | — | 5:4:45_{(H2O)} | 37 |
| DMSP HCl | glycerol | — | 1:2:5_{(H2O)} | 20 |
| glycerol | sorbitol | — | 2:1 | — |
| proline | guanidine HCl | — | 2:1:8_{(H2O)} | 30 |
| proline | arginine | — | 2:1:11_{(H2O)} | 33 |
| ectoine | urea | — | 1:2:2_{(H2O)} | 12 |
| ectoine | guanidine HCl | — | 1:2:3_{(H2O)} | 14 |
| ectoine | arginine | — | 2:1:10_{(H2O)} | 30 |
| ectoine | glycerol | — | 1:2:45_{(H2O)} | 20 |
| sorbitol | urea | — | 2:3:1_{(H2O)} | 3 |
| sorbitol | guanidine HCl | — | 2:3:1_{(H2O)} | 3 |
| sucrose | urea | — | 4:1:8_{(H2O)} | 9 |
| sucrose | guanidine HCl | — | 2:1:12_{(H2O)} | 23 |
| sucrose | arginine | — | 5:2:16_{(H2O)} | 12 |
| betaine | sorbitol | guanidine | 1:1.2:1:7_{(H2O)} | 23 |
| | | HCl | | |
| betaine | sorbitol | glycerol | 1:1:2 | — |
| betaine | taurine | glycerol | 1:1:3 | — |
| betaine | ectoine | glycerol | 1:2:3 | — |
| betaine | ectoine | sorbitol | 1:2:3:5_{(H2O)} | 9 |
| TMAO | urea | glycerol | 1:2:2:2_{(H2O)} | 8 |
| TMAO | guanidine HCl | glycerol | 1:1:2:2_{(H2O)} | 9 |
| TMAO | urea | sorbitol | 1:2:2:2_{(H2O)} | 6 |
| TMAO | guanidine HCl | sorbitol | 1:1:2:2_{(H2O)} | 6 |
| TMAO | betaine | urea | 1:1:2:2_{(H2O)} | 10 |
| TMAO | betaine | guanidine | 1:1:2:6_{(H2O)} | 21 |
| | | HCl | | |
| fructose | glucose | guanidine | 1:1:2:1 | 4 |
| | | HCl | | |
| fructose | glycerol | guanidine | 2:1:1:1_{(H2O)} | 4 |
| | | HCl | | |
| glucose | glycerol | guanidine | 2:1:1:6_{(H2O)} | 16 |
| | | HCl | | |
| glucose | trehalose | urea | 1:1:2:3_{(H2O)} | 7 |
| glucose | trehalose | guanidine | 1:1:1:3_{(H2O)} | 8 |
| | | HCl | | |
| sorbitol | glycerol | guanidine | 1:1:2 | — |
| | | HCl | | |
| trehalose | glucose | glycerol | 1:1:5 | — |
| trehalose | glucose | sorbitol | 1:1:4:13_{(H2O)} | 20 |
| trehalose | proline | glycerol | 1:1:4:8_{(H2O)} | 15 |
| trehalose | ectoine | glycerol | 1:1:4:6_{(H2O)} | 11 |
| proline | glycerol | sorbitol | 1:1:1:3_{(H2O)} | 12 |

In the most preferred embodiments the multicomponent DESs that comprise at least four components comprise the components in combinations and ratios as given in the Table 5 below.

TABLE 5
Exemplary embodiments of the multi-component deep eutectic
solvents comprising naturally occurring osmolytes

| | | | | | | | Water |
|---|---|---|---|---|---|---|---|
| Components | | | | | | | content |
| 1 | 2 | 3 | 4 | 5 | 6 | Components molar ratio | (wt. %) |
| Betaine | GPC | Sorbitol | Taurine | Urea | — | 1:2.8:3.1:0.1:7.1:1_{(H2O)} | 7 |
| Betaine | GPC | Sorbitol | Inositol | Urea | — | 1:0.7:0.7:0.9:7.1:3.5_{H2O)} | 38 |
| Betaine | GPC | Sorbitol | Inositol | Urea | — | 1:0.9:1.2:0.6:8.1:3_{(H2O)} | 30 |
| Betaine | GPC | Sorbitol | Inositol | Urea | — | 1:0.9:1.5:0.4:8.3:2.5_{(H2O)} | 21 |
| Betaine | Glycerol | Sorbitol | Ectoine | — | — | 1:4:2:1:14_{(H2O)} | 20 |
| TMAO | Betaine | Taurine | Urea | — | — | 1:0.1:0.1:1.5:5_{(H2O)} | 34 |
| TMAO | Betaine | Taurine | Urea | — | — | 1:6:0.5:15:9_{(H2O)} | 9 |
| TMAO | Betaine | Sorbitol | α-alanine | Taurine | Urea | 1:0.1:0.05:0.1:0.05:2:2_{(H2O} | 14 |
| TMAO | Betaine | Inositol | α-alanine | Urea | — | 1:0.1:0.05:0.2:1:5_{(H2O)} | 37 |
| TMAO | Inositol | Urea | α-alanine | — | — | 1:0.05:1:0.2:5_{(H2O)} | 37 |
| Trehalose | Proline | Glycerol | Sorbitol | — | — | 1:1:4:1:14_{(H2O)} | 20 |

### EXAMPLES

### Example 1: Preparation of Exemplary DESs According to an Embodiment

In order to test the prepared DESs according to an embodiment as well as to show their capacity to stabilize biomolecules, a group of multi-component DESs was prepared according to the combinations and ratios given in the table 6 below.

TABLE 6
Multiple-component DESs

| | | | Water |
|---|---|---|---|
| | | Components | content |
| DES | Components | molar ratio | (wt. %) |
| ChCl:Gly | ChCl:Glycerol | 1:2 | 20 |
| | | | 40 |
| Bet:U | Betaine:Urea | 1:1 | 20 |
| TMAO:Gly | TMAO:Glycerol | 1:2 | 40 |
| Sar:Gly | Sarcosine:Glycerol | 1:2 | 20 |
| | | | 40 |
| DMPS:Gly | DMSP:Glycerol | 1:2 | 20 |
| | | | 40 |
| Ect:Gly | Ectoine:Glycerol | 1:2 | 20 |
| | | | 40 |
| Bet:Sor:Tau:GPC:U | Betaine:Sorbitol:Taurine:GPC:Urea | 1:3.1:0.1:2.8:7.1 | 20 |
| TMAO:Bet:Tau:U | TMAO:Betaine:Taurine:Urea | 1:0.1:0.1: 1.5 | 20 |
| Bet:Gly:Sor | Betaine:Glycerol:Sorbitol | 1:1:2 | 20 |
| Bet:Gly:Sor:Ect | Betaine:Glycerol:Sorbitol:Ectoine | 1:4:2:1 | 20 |
| Treh:Pro:Gly:Sor | Trehalose:Proline:Glycerol:Sorbitol | 1:1:4:1 | 20 |
| Treh:Pro:Gly:Sor | Trehalose:Proline:Glycerol:Sorbitol | 1:1:4:1 | 40 |

The DESs were prepared by calculating the appropriate amount of each component, adding it to a mixture and heating at a temperature up to 60 °C to obtain a liquid solution that was used in further experiments.

### Example 2: Lysozyme Stabilization at 25 °C by Selected DESs Prepared in Example 1

Lysozyme activity was determined according to the method of Shugar et al. (1952, Biochim Biophys Acta 8:302-9), which is based on the decrease in turbidity of the substrate, Micrococcus lysodeikticus, in a suspension. Lysozyme solutions at a concentration of 0.1 mg ml⁻¹ were prepared in different DESs and in 10 mM sodium phosphate buffer solution (pH 7). The solutions were incubated at different temperatures (25, 45, 70 °C) for 1, 3, 5 and 7 days (Examples 2 and 3) or 15, 60 and 120 min (Example 4).

Briefly, to 525 μl of 10 mM sodium phosphate buffer solution (pH 7) in a plastic disposable cuvette 30 μl of Micrococcus lysodeikticus bacteria suspension in sterile PBS buffer (7 mg ml⁻¹) and 30 μl of the lysozyme solution were added. Immediately after mixing, the cuvette was placed in a UV/VIS spectrophotometer and the absorbance was measured at a wavelength of 450 nm over a period of linear turbidity decline. The relative activity (%) was calculated from the initial reaction rate obtained by the enzyme (calculated from ΔA450 min⁻¹) after incubation, compared to the one obtained without previous exposure.

TABLE 7
Incubation of lysozyme in different DES and 10 mM sodium phosphate buffer
solution (pH 7) at 25 °C for 1, 3, 5 and 7 days. Results are presented
as residual activity - relative to the enzyme activity prior incubation (%).

| | Water | Residual activity (%) after incubation for | | | |
|---|---|---|---|---|---|
| DESs | content | 1 day | 3 days | 5 days | 7 days |
| Control 1 (phosphate | | 89.4 | 78.4 | 77.2 | 75.4 |
| buffer) | | | | | |
| Control 2: Stabilizing | 20 | 95.3 | 86.6 | 85.3 | 81.6 |
| medium for lysozyme | | | | | |
| (ChCl:Gly 1:2) | | | | | |
| Sar:Gly 1:2 | 20 | 97.3 | 84.1 | 90.3 | 95.8 |
| Sar:Gly 1:2 | 40 | 102.9 | 93.3 | 100.2 | 108.8 |
| DMSP:Gly 1:2 | 20 | 92.2 | 95.4 | 100.0 | 95.3 |
| Ectoine:Gly 1:2 | 40 | 99.2 | 102.9 | 103.6 | 109.8 |
| Bet:Sor:Tau:GPC:U | 20 | NA | 94.9 | 95.3 | 96.2 |
| Bet:Gly:Sor | 20 | NA | 91.8 | 92.4 | 93.2 |
| Bet:Gly:Sor:Ect | 20 | NA | 110.0 | 105.4 | 101.5 |

NA = not analyzed

As can be seen from the table, DESs comprising sarcosine and glycerol in any water content enabled higher residual lysozyme activity in comparison to both controls, especially after 5 and 7 days. The effect was more pronounced in the composition with higher water content. Similar values as for Sar: Gly 40 were observed for the DESs containing DMSP and glycerol, the DESs containing betaine, sorbitol, GPC, urea and taurine, and the DESs containing betaine, sorbitol and glycerol. Presence of ectoine is, however, the most preferred for lysozyme stabilization, as all analysed DESs comprising ectoine showed more than 100% residual activity, even after one week. These results suggest that the prepared DESs in all their variability stabilize lysozyme stored at 25 °C in a significantly better and longer-lasting manner than the currently known best stabilizing medium containing ChCl and glycerol.

### Example 3: Lysozyme Stabilization at 45 °C by Selected DESs Prepared in Example 1

The experiment was performed in the same manner as described for Example 2, except that the incubation temperature was 45 °C.

TABLE 8
Incubation of lysozyme in different DES and 10 mM sodium phosphate buffer
solution (pH 7) at 45 °C for 1, 3, 5 and 7 days. Results are presented
as residual activity - relative to the enzyme activity prior incubation (%).

| | Water | Residual activity (%) after incubation for | | | |
|---|---|---|---|---|---|
| DESs | content | 1 day | 3 days | 5 days | 7 days |
| Control 1 (phosphate | | 66.3 | 55.9 | 50.3 | 50.2 |
| buffer) | | | | | |
| Control 2 (ChCl:Gly 1:2) | 20 | 85.4 | 79.7 | 81.2 | 79.3 |
| Sar:Gly | 20 | 99.5 | 102.4 | 99.4 | 103.3 |
| Sar:Gly | 40 | 102.7 | 115.4 | 114.3 | 106.7 |
| DMSP:Gly | 20 | 93.3 | 100.3 | 102.4 | 106.7 |
| Ectoine:Gly | 40 | 99.2 | 99.7 | 102.4 | 105.0 |
| Bet:Sor:Tau:GPC:U | 20 | NA | 89.7 | 88.3 | 86.2 |
| Bet:Gly:Sor | 20 | NA | 130.8 | 110.3 | 108.2 |
| Bet:Gly:Sor:Ect | 20 | NA | 99.5 | 96.3 | 86.7 |

The results presented in table 8 indicate that all DESs enabled significantly higher residual lysozyme activity in comparison to both controls, even after 1 day, but especially after 5 and 7 days. Many of residual activities were higher than 100%, in accordance with previous observations (Delorme et al., 2020: doi: 10.1016/j.ijbiomac.2020.07.022; Kaar et al., 2022; J. AM. CHEM. SOC. 2003, 125, 4125-4131; Varriale et al., 2022:10.1021/acssuschemeng.1c07104) Overall, these results suggest that the prepared DESs in all their variability stabilize lysozyme stored at 45 °C in a significantly better and longer-lasting manner than the currently known best stabilizing medium containing ChCl and glycerol.

### Example 4: Lysozyme Stabilization at 70 °C by Selected DESs Prepared in Example 1

The experiment was performed in the same manner as described for Example 2, except that the incubation temperature was 70 °C and incubation times were 15, 60 and 120 minutes, respectively.

TABLE 9
Incubation of lysozyme in different DES and 10 mM sodium phosphate buffer solution
(pH 7) at 70 °C (heat shock) for 15-, 60- or 120-min. Results are presented
as residual activity - relative to the enzyme activity prior incubation (%).

| | | Residual activity (%) after incubation for | | |
|---|---|---|---|---|
| | Water | 15 | 60 | 120 |
| DESs | content | minutes | minutes | minutes |
| Control 1 (phosphate | | 83.9 | 69.2 | 59.8 |
| buffer) | | | | |
| Control 2 (ChCl:Gly 1:2) | 20 | 87.2 | 79.7 | 69.6 |
| TMAO: Gly | 40 | 109.2 | 105.5 | 78.9 |
| Sar:Gly | 20 | 84.4 | 83.0 | 80.3 |
| Sar:Gly | 40 | 103.8 | 102.9 | 100.3 |
| DMSP:Gly | 20 | 106.8 | 103.3 | 103.4 |
| Ectoine:Gly | 40 | 112.7 | 110.6 | 96.4 |
| Bet:Sor:Tau:GPC:U | 20 | 94.2 | 87.9 | 85.2 |
| Bet:Gly:Sor | 20 | 110.1 | 115.0 | 105.6 |
| Bet:Gly:Sor:Ect | 20 | 84.6 | 77.1 | 77.1 |

The results presented in table 9 suggest that all DESs enabled significantly higher resistance of lysozyme to heat shock in comparison to both controls. Many of residual activities were higher than 100%. The pronounced stabilization effect was least significant for the DESs containing sarcosine and glycerol as well as for the DESs consisting of betaine, glycerol, sorbitol and ectoine. These results suggest that the prepared DESs in all their variability stabilize lysozyme treated at 70 °C in a significantly better manner than the currently known media, whereas some of DESs exhibit better function at lower temperatures, while some are better for protection against heat shock.

### Example 5: Yeast Alcohol Dehydrogenase (YADH) Stability by Selected DESs Prepared in Example 1

YADH activity was determined according to the method of Walker et al. (Doi: 10.1016/0307-4412(92)90021-D). YADH solutions at a concentration of 0.4 mg ml⁻¹ were prepared in different DESs and in glycine-pyrophosphate buffer (pH 9). The solutions were incubated at different temperatures (25 and 45 °C) for 2, 5, 8, 24 and 48 h (at 25 °C) or 1, 2, 4, 8 and 24 h (at 45 °C).

Briefly, to 975 μl of glycine-pyrophosphate buffer (pH 9) in a plastic disposable cuvette 5 μl of NAD⁺ in PBS buffer (50 mg ml⁻¹), 10 μl of ethanol (96%) and 10 μl of the YADH solution were added. Immediately after mixing, the cuvette was placed in a UV/VIS spectrophotometer and the absorbance was measured at a wavelength of 340 nm for 2 minutes. The relative activity (%) was calculated from the initial reaction rate obtained by the enzyme (calculated from ΔA340 min⁻¹) after incubation, compared to the one obtained without previous exposure.

TABLE 10
Incubation of YADH in different DES and 10 mM sodium phosphate
buffer solution (pH 7) at 25 °C for 2, 5, 8, 24 and 48
hours, respectively. Results are presented as residual activity -
relative to the enzyme activity prior incubation (%).

| | | Residual activity (%) after | | | | |
|---|---|---|---|---|---|---|
| | Water | incubation at 25 °C for | | | | |
| DESs | content | 2 h | 5 h | 8 h | 24 h | 48 h |
| Control 1 (phosphate | | 95.5 | 51.5 | 34.0 | 8.4 | 0 |
| buffer) | | | | | | |
| Control 2 (ChCl:Gly 1:2) | 40 | 55.0 | 50.8 | 2.17 | 0 | 0 |
| Sar:Gly | 20 | 112.6 | 123.9 | 110.2 | 50.7 | 95.2 |
| Sar:Gly | 40 | 76.6 | 96.7 | 105.5 | 98.2 | 95.2 |
| Treh:Ect:Gly | 20 | 80.9 | 90.5 | 82.8 | 26.8 | 0 |
| Treh:Ect:Gly | 40 | 135.0 | 123.2 | 78.6 | 13.6 | 17.2 |
| Treh:Pro:Gly:Sor | 20 | 77.6 | 71.4 | 62.1 | 51.1 | 57.8 |
| Treh:Pro:Gly:Sor | 40 | 83.3 | 78.5 | 63.3 | 47.6 | 0 |

TABLE 11
Incubation of YADH in different DES and 10 mM sodium phosphate
buffer solution (pH 7) at 45 °C for 1, 2, 4, 8, and 24
hours, respectively. Results are presented as residual activity -
relative to the enzyme activity prior incubation (%).

| | | Residual activity (%) after | | | | |
|---|---|---|---|---|---|---|
| | Water | incubation at 45 °C for | | | | |
| DESs | content | 1 h | 2 h | 4 h | 8 h | 24 h |
| Control 1 (phosphate | | 0 | 0 | 0 | 0 | 0 |
| buffer) | | | | | | |
| Control 2 (ChCl:Gly 1:2) | 20 | 2.1 | 0 | 0 | 0 | 0 |
| | 40 | 1.7 | | | | |
| Sar:Gly | 20 | 136.9 | 138.6 | 66.9 | 34.4 | 11.3 |
| Sar:Gly | 40 | 93.8 | 88.8 | 72.7 | 40.6 | 4.2 |
| Treh:Ect:Gly | 40 | 39.8 | 3.5 | 0 | 0 | 0 |
| Bet:Sor:Tau:GPC:U | 20 | 25.0 | 2.6 | 0 | 0 | 0 |

The results shown in tables 10 and 11 show that some DESs according to an embodiment can stabilize YADH to a higher degree than conventionally used buffers or stabilizing media. Among tested DES, exceptional stabilisation properties of Sar: Gly have been observed.

Based on known results of other DES stabilization of other proteins as well as DNA and other biomolecules is expected. Due to presence of osmolytes in the DESs according to embodiments disclosed herein, cryoprotection of cells and tissues may be enabled by these embodiments.

Changes and modifications in the specifically described embodiments can be carried out without departing from the principles of the present invention which is intended to be limited only by the scope of the appended claims, as interpreted according to the principles of patent law including the doctrine of equivalents.

1. A multicomponent deep eutectic solvent (DESs) comprising at least four components.

2. The multicomponent DESs according to claim 1, wherein at least one of the components of the DESs are selected in the group comprising naturally occurring osmolytes.

3. The multicomponent DESs according to claim 2, wherein
the naturally occurring osmolytes are selected in the group comprising:
polyols and sugar polyols, for example glycerol, sorbitol, xylitol;
sugars and their derivatives, for example glucose, sucrose, trehalose;
amino acids and their derivatives, for example glycine, proline, ectoine, taurine;
methylamines, for example trimethylamine N-oxide (TMAO), sarcosine, betaine, glycerophosphocholine (GPC);
methylsulfonium compounds, for example dimethylsulfoniopropionate (DMSP);
Y-conjugated compounds, for example urea, guanidine and arginine.

4. A multicomponent deep eutectic solvent comprising at least two components, wherein at least one of the components is selected in the group consisting of ectoine, trimethylamine N-oxide (TMAO), sarcosine, glycerophosphocholine (GPC), dimethylsulfonopropionate (DMSP), guanidine, arginine, and taurine.

5. The multicomponent DESs according to claim 4, wherein the multicomponent DESs has at least three, preferably at least four components.

6. The multicomponent DESs according to claim 1, wherein remaining components of the multicomponent DESs are selected among common DES/IL compounds, such as methylamines, methylsulfonium compounds, organic acids, sugars, polyols, amino acids and Y-conjugated compounds.

7. The multicomponent DESs according to claim 6, wherein methylamines are selected from N-trimethylamine oxide (TMAO), betaine, glycerophosphocholine (GPC), carnitine, homarine, and derivatives thereof, for example, their halide forms, such as betaine halides (betaine HCl).

8. The multicomponent DESs according to claim 6, wherein methyl sulfonium compound is dimethylsulfonopropionate (DMSP).

9. The multicomponent DESs according to claim 6, wherein organic acids are selected in the group comprising: levulinic acid, lactic acid, malic acid, maleic acid, pyruvic acid, fumaric acid, succinic acid, citric acid, citraconic acid, glutaric acid, glycolic acid, acetic acid, aconitic acid, tartaric acid, ascorbic acid, malonic acid, oxalic acid, glucuronic add, neuraminic acid, sialic acid, shikimic acid, phytic acid, galacturonic acid, iduronic acid, hyaluronic acid, hydroxycitric acid, lactone derivatives and derivatives thereof.

10. The multicomponent DESs according to claim 6, wherein sugars are selected in the group comprising: trehalose, glucose, sucrose, lactose, ribose, galactose, fructose, etc. and derivatives thereof.

11. The multicomponent DESs according to claim 6, wherein polyols are selected in the group comprising: glycerol, erythritol, mannitol, sorbitol, xylitol, ethylene glycol, propylene glycol, ribitol, aldonitol, propanediol, inositol, pentylene glycol, and derivatives thereof, such as o-methyl-inositol.

12. The multicomponent DESs according to claim 6, wherein amino acids are selected in the group comprising: glycine, proline, taurine, lysine, etc. and derivatives thereof, such as ectoine, sarcosine, theanine, dimethyglycine and similar.

13. The multicomponent DESs according to claim 6, wherein Y-conjugated compounds are selected in the group comprising: urea, guanidine, arginine and compounds that incorporate the guanidino moiety, such as creatine, glycocyamine, agmatine, 4-guanidinobutanoic acid, and derivatives thereof, such as guanidinium halides, for example guanidine HCl.

14. The multicomponent DESs according to claim 1, wherein the molar ratio of each DES component with respect to the total moles of the DES may range from 0.001 to 0.9.

15. The multicomponent DESs according to claim 1, wherein the DESs also comprises water.

16. The multicomponent DESs according to claim 15, wherein the amount of water used in the DESs is from 0% to 90%, as from about 0.00001, 0.0001, 0.001, 0.01, 0.1, 0.5, 1, 5, 10, 15, 20, 30 or about 40% to about 90, 80, 70, 60 or about 50%.

17. The multicomponent DESs according to claim 16, wherein the amount of water used in from about 5% to about 30%, such as about 10%, 20% 25% or about 30%.

18. (canceled)

19. The multicomponent DESs according to claim 1, wherein the multicomponent DESs is selected in the group consisting of:
Components

| 1 | 2 | 3 | Water |
|---|---|---|---|
| betaine | arginine | — | yes |
| betaine | guanidine HCl | — | yes |
| TMAO | urea | — | yes |
| TMAO | glycerol | — | yes |
| TMAO | sorbitol | — | yes |
| TMAO | glucose | — | yes |
| TMAO | trehalose | — | yes |
| TMAO | xylitol | — | yes |
| TMAO | guanidine HCl | — | yes |
| TMAO | arginine | — | yes |
| sarcosine | urea | — | yes |
| sarcosine | guanidine HCl | — | yes |
| sarcosine | arginine | — | yes |
| sarcosine | glycerol | — | yes |
| GPC | urea | — | yes |
| GPC | guanidine HCl | — | yes |
| GPC | arginine | — | yes |
| DMSP HCl | urea | — | yes |
| DMSP HCl | guanidine HCl | — | yes |
| DMSP HCl | arginine | — | yes |
| DMPS HCl | glycerol | — | yes |
| glycerol | sorbitol | — | no |
| proline | guanidine HCl | — | yes |
| proline | arginine | — | yes |
| ectoine | urea | — | yes |
| ectoine | guanidine HCl | — | yes |
| ectoine | arginine | — | yes |
| ectoine | glycerol | — | yes |
| sorbitol | urea | — | yes |
| sorbitol | guanidine HCl | — | yes |
| sucrose | urea | — | yes |
| sucrose | guanidine HCl | — | yes |
| sucrose | arginine | — | yes |
| betaine | sorbitol | guanidine HCl | yes |
| betaine | taurine | glycerol | no |
| betaine | ectoine | glycerol | no |
| betaine | ectoine | sorbitol | yes |
| TMAO | urea | glycerol | yes |
| TMAO | guanidine HCl | glycerol | yes |
| TMAO | urea | sorbitol | yes |
| TMAO | guanidine HCl | sorbitol | yes |
| TMAO | betaine | urea | yes |
| TMAO | betaine | guanidine HCl | yes |
| fructose | glucose | guanidine HCl | yes |
| fructose | glycerol | guanidine HCl | yes |
| glucose | glycerol | guanidine HCl | yes |
| glucose | trehalose | urea | yes |
| glucose | trehalose | guanidine HCl | yes |
| sorbitol | glycerol | guanidine HCl | no |
| trehalose | glucose | glycerol | no |
| trehalose | glucose | sorbitol | yes |
| trehalose | proline | glycerol | yes |
| trehalose | ectoine | glycerol | yes |
| proline | glycerol | sorbitol | yes |

20. The multicomponent DESs according to claim 1, wherein the multicomponent DESs is selected in the group consisting of:

| | | | | Water |
|---|---|---|---|---|
| Components | | | Components | content |
| 1 | 2 | 3 | molar ratio | (wt. %) |
| betaine | arginine | — | 5:1:28_{(H2O)} | 40 |
| betaine | guanidine HCl | — | 1:2:3_{(H2O)} | 17 |
| TMAO | urea | — | 1:1:3.5_{(H2O)} | 33 |
| TMAO | glycerol | — | 1:1:2_{(H2O)} | 17 |
| TMAO | sorbitol | — | 1:1:2_{(H2O)} | 12 |
| TMAO | glucose | — | 5:2:12_{(H2O)} | 23 |
| TMAO | trehalose | — | 4:1:8_{(H2O)} | 18 |
| TMAO | xylitol | — | 1:1:4_{(H2O)} | 24 |
| TMAO | guanidine HCl | — | 1:1:2_{(H2O)} | 18 |
| TMAO | arginine | — | 15:4:56_{(H2O)} | 37 |
| sarcosine | urea | — | 2:5:9_{(H2O)} | 25 |
| sarcosine | guanidine HCl | — | 2:5:11_{(H2O)} | 23 |
| sarcosine | arginine | — | 3:2:19_{(H2O)} | 36 |
| sarcosine | glycerol | — | 1:2:4_{(H2O)} | 20 |
| GPC | urea | — | 1:2:1.5_{(H2O)} | 7 |
| GPC | guanidine HCl | — | 1:2:1_{(H2O)} | 4 |
| GPC | arginine | — | 11:4:80_{(H2O)} | 30 |
| DMSP HCl | urea | — | 1:2:2_{(H2O)} | 11 |
| DMSP HCl | guanidine HCl | — | 1:1:3_{(H2O)} | 17 |
| DMSP HCl | arginine | — | 5:4:45_{(H2O)} | 37 |
| DMSP HCl | glycerol | — | 1:2:5_{(H2O)} | 20 |
| glycerol | sorbitol | — | 2:1 | — |
| proline | guanidine HCl | — | 2:1:8_{(H2O)} | 30 |
| proline | arginine | — | 2:1:11_{(H2O)} | 33 |
| ectoine | urea | — | 1:2:2_{(H2O)} | 12 |
| ectoine | guanidine HCl | — | 1:2:3_{(H2O)} | 14 |
| ectoine | arginine | — | 2:1:10_{(H2O)} | 30 |
| ectoine | glycerol | — | 1:2:4.5_{(H2O)} | 20 |
| sorbitol | urea | — | 2:3:1_{(H2O)} | 3 |
| sorbitol | guanidine HCl | — | 2:3:1_{(H2O)} | 3 |
| sucrose | urea | — | 4:1:8_{(H2O)} | 9 |
| sucrose | guanidine HCl | — | 2:1:12_{(H2O)} | 23 |
| sucrose | arginine | — | 5:2:16_{(H2O)} | 12 |
| betaine | sorbitol | guanidine HCl | 1:1.2:1:7_{(H2O)} | 23 |
| betaine | sorbitol | glycerol | 1:1:2 | — |
| betaine | taurine | glycerol | 1:1:3 | — |
| betaine | ectoine | glycerol | 1:2:3 | — |
| betaine | ectoine | sorbitol | 1:2:3:5_{(H2O)} | 9 |
| TMAO | urea | glycerol | 1:2:2:2_{(H2O)} | 8 |
| TMAO | guanidine HCl | glycerol | 1:1:2:2_{(H2O)} | 9 |
| TMAO | urea | sorbitol | 1:2:2:2_{(H2O)} | 6 |
| TMAO | guanidine HCl | sorbitol | 1:1:2:2_{(H2O)} | 6 |
| TMAO | betaine | urea | 1:1:2:2_{(H2O)} | 10 |
| TMAO | betaine | guanidine HCl | 1:1:2:6_{(H2O)} | 21 |
| fructose | glucose | guanidine HCl | 1:1:2:1 | 4 |
| fructose | glycerol | guanidine HCl | 2:1:1:1_{(H2O)} | 4 |
| glucose | glycerol | guanidine HCl | 2:1:1:6_{(H2O)} | 16 |
| glucose | trehalose | urea | 1:1:2:3_{(H2O)} | 7 |
| glucose | trehalose | guanidine HCl | 1:1:1:3_{(H2O)} | 8 |
| sorbitol | glycerol | guanidine HCl | 1:1:2 | — |
| trehalose | glucose | glycerol | 1:1:5 | — |
| trehalose | glucose | sorbitol | 1:1:4:13_{(H2O)} | 20 |
| trehalose | proline | glycerol | 1:1:4:8_{(H2O)} | 15 |
| trehalose | ectoine | glycerol | 1:1:4:6_{(H2O)} | 11 |
| proline | glycerol | sorbitol | 1:1:1:3_{(H2O)} | 12 |

21. The multicomponent DESs according to claim 1, wherein the multicomponent DESs is selected in the group consisting of:

| | | | | | | | Water |
|---|---|---|---|---|---|---|---|
| Components | | | | | | | content |
| 1 | 2 | 3 | 4 | 5 | 6 | Components molar ratio | (wt. %) |
| Betaine | GPC | Sorbitol | Taurine | Urea | — | 1:2.8:3.1:0.1:7.1:1_{(H2O)} | 7 |
| Betaine | GPC | Sorbitol | Inositol | Urea | — | 1:0.7:0.7:0.9:7.1:3.5_{H2O)} | 38 |
| Betaine | GPC | Sorbitol | Inositol | Urea | — | 1:0.9:1.2:0.6:8.1:3_{(H2O)} | 30 |
| Betaine | GPC | Sorbitol | Inositol | Urea | — | 1:0.9:1.5:0.4:8.3:2.5_{(H2O)} | 21 |
| Betaine | Glycerol | Sorbitol | Ectoine | — | — | 1:4:2:1:14_{(H2O)} | 20 |
| TMAO | Betaine | Taurine | Urea | — | — | 1:0.1:0.1:1.5:5_{(H2O)} | 34 |
| TMAO | Betaine | Taurine | Urea | — | — | 1:6:0.5:15:9_{(H2O)} | 9 |
| TMAO | Betaine | Sorbitol | α-alanine | Taurine | Urea | 1:0.1:0.05:0.1:0.05:2:2_{(H2O} | 14 |
| TMAO | Betaine | Inositol | α-alanine | Urea | — | 1:0.1:0.05:0.2:1:5_{(H2O)} | 37 |
| TMAO | Inositol | Urea | α-alanine | — | — | 1:0.05:1:0.2:5_{(H2O)} | 37 |
| Trehalose | Proline | Glycerol | Sorbitol | — | — | 1:1:4:1:14_{(H2O)} | 20 |

22. (canceled)

23. Use of the multi-component DESs according to claim 1 in:
stabilization of biomolecules,
preservation of biological systems,
pharmaceutical preparations, such as pharmaceutical excipients and similar components,
cosmetics,
food industry, and
preparation of extracts, wherein the DESs is optionally a component of the final extract.

24. (canceled)

25. (canceled)

26. A medium for stabilization of biomolecules or preservation biological systems comprising the DESs according to claim 1, wherein the biomolecule is selected in the group comprising, cell components, proteins, enzymes, antibodies, peptides and other molecules consisting of aminoacids or aminoacid derivatives, polysaccharides, biopolymers, lipids, supramolecular structures containing lipids, nucleotides, nucleosides and their precursors and derivatives, polynucleotides of any length, sense and molecular weight and composition, DNA and RNA.

27. A method for stabilization of biomolecules or for preservation of biological systems comprises at least a step in which the biomolecule or the biological system are exposed to a medium for stabilization of biomolecules or preservation biological systems comprising the DESs according to claim 1.

28. The method according to claim 27, wherein the biological system is an organism, an organ, an organelle, a cell or a tissue or an engineered construct.

29. The method according to claim 27, wherein the biomolecule is selected in the group comprising, cell components, proteins, enzymes, antibodies, peptides and other molecules consisting of amino acids or amino acid derivatives, polysaccharides, biopolymers, lipids, supramolecular structures containing lipids, nucleotides, nucleosides and their precursors and derivatives, polynucleotides of any length, sense and molecular weight and composition, DNA and RNA.
